(19) United States
(12) Patent Application Publication
(10) Pub. No.: US 2007/0202547 A1
(43) Pub. Date: Aug. 30, 2007

(54) Methods For Detecting Substances Which Bind To The Amyloid Precursor Protein Or Beta Amyloid Fragments, And Binding Compounds

(76) Inventors: Craig A. Coburn, Royersford, PA (US); Amy S. Espeseth, Chalfont, PA (US); Daria J. Hazuda, Doylestown, PA (US)

Correspondence Address: MERCK AND CO., INC, P O BOX 2000, RAHWAY, NJ, 07065-0907, US

(21) Appl. No.: 10/594,835

(22) PCT Filed: Mar. 29, 2005
(86) PCT No.: PCT/US05/10538

§ 371 Date: Sep. 28, 2006

Related U.S. Application Data
(60) Provisional application No. 60/558,855, filed on Apr. 2, 2004.
(60) Provisional application No. 60/588,185, filed on Jul. 15, 2004.

(51) Int. Cl. G01N 33/542

(52) U.S. Cl. 435/7.9

(57) ABSTRACT

Disclosed are methods for identifying and evaluating the binding properties of substances to the amyoid precursor protein (APP) or to β-amyloid (Aβ fragments of APP. Also disclosed is a class of benzofuran derivatives of formula (I), which interact specifically with APP or Aβ, and block interaction of APP or Aβ with secreatase or APP or Aβ binding antibodies.

### CROSS-REFERENCE TO RELATED APPLICATIONS

This application claims priority under 35 U.S.C. § 119(e) of U.S. provisional applications Ser. No. 60/558,855, filed Apr. 7, 2004 and Ser. No. 60/588,185, filed Jul. 15, 2004.

### FIELD OF THE INVENTION

The present invention is directed generally to the field of neurological and physiological dysfunctions associated with Alzheimer's Disease (“AD”). More particularly, the invention is directed to methods for identifying and evaluating the binding properties of substances to the amyloid precursor protein (APP), or to β-amyloid (Aβ) fragments of APP. The invention is also directed to a class of benzofuran derivatives which interact specifically with APP or Aβ, and block interaction of APP or Aβ with secretases or with APP or Aβ binding antibodies.

### BACKGROUND OF THE INVENTION

Alzheimer's Disease is a common neurodegenerative disease affecting the elderly. The key features of the disease include progressive memory impairment, loss of language and visuospatial skills, and behavior deficits. These changes in cognitive function are the result of degeneration of neurons in the cerebral cortex, hippocampus, basal forebrain, and other regions of the brain. Accumulation of a 39-43 amino acid peptide, termed amyloid β peptide (Aβ), is believed to be a cause of Alzheimer's Disease. Aβ is produced in the brain by processing of the beta-amyloid precursor protein (APP) by the beta-amyloid protein cleaving enzyme (“beta secretase” or “BACE”) and gamma-secretase. The processing leads to accumulation of Aβ in the brain.

Much of the recent progress in elucidating the pathogenesis of AD has centered on the apparent role of Aβ as a unifying pathological feature of the genetically diverse forms of this complex disorder. The defining neuropathological characteristic of AD is the accumulation of insoluble proteinaceous deposits, known as amyloid plaques, in the brains of those affected. The presence of these amyloid plaque deposits is the essential observation underpinning the amyloid hypothesis.

Evidence suggests that deposition of AD plays a significant role in the development of amyloid plaques and the etiology of AD. For example, individuals with mutations in the gene encoding APP (from which the Aβ protein is derived) invariably develop Alzheimer's disease (Goate et al., 1991, Nature 353:844-846; Mullan et al., 1992, Nature Genet. 1:345-347; Murrell et al., 1991, Science 254:97-99; Van Broecknoven, 1995, Eur. J. Neurol. 35:8-19). Likewise, autopsies have shown that amyloid plaques are found in the brains of virtually all Alzheimer's patients and that the degree of amyloid plaque deposition correlates with the degree of dementia (Cummings & Cotman, 1995, Lancet 326:1524-1587).

While abundant evidence suggests that extracellular accumulation and deposition of Aβ is a central event in the etiology of AD, recent studies have also proposed that increased intracellular accumulation of Aβ or amyloid containing C-terminal fragments (CTFβ) may play a role in the pathophysiology of AD. For example, over-expression of APP harboring mutations which cause familial AD results in the increased intracellular accumulation of CTFβ in neuronal cultures and AβD42 in HEK 293 cells. Aβ42 is the 42 amino acid long form of Aβ that is believed to be more efficacious at forming amyloid plaques than shorter forms of Aβ. Moreover, evidence suggests that intra- and extracellular Aβ are formed in distinct cellular pools in hippocampal neurons, and increased intracellular accumulation of Aβ42 is a common feature associated with two types of familial AD mutations in APP (“Swedish” and “London”). Thus, based on these studies and earlier reports implicating extracellular Aβ accumulation in AD pathology, it appears that altered APP catabolism may be involved in disease progression.

APP is an ubiquitous membrane-spanning (type 1) glycoprotein that undergoes a variety of proteolytic processing events. (Selkoe, 1998, Trends Cell Biol. 8:447-453). APP is actually a family of peptides produced by alternative splicing from a single gene. Major forms of APP are known as APP₆₉₅, APP₇₅₁, and APP₇₇₀, with the subscripts referring to the number of amino acids in each splice variant (Ponte et al., 1988, Nature 331:525-527; Tanzi et al., 1988, Nature 331:528-530; Kitaguchi et al., 1988, Nature 331:530-532). APP is expressed and constitutively catabolized in most cells.

APP has a receptor-like structure with a large ectodomain, a membrane spanning region and a short cytoplasmic tail. The Aβ domain encompasses parts of both extra-cellular and transmembrane domains of APP. Release of Aβ from APP implies the existence of two distinct proteolytic events to generate the NH₂— and COOH-termini of Aβ. At least two secretory mechanisms exist which release APP from the membrane and generate soluble, COOH-truncated forms of APP (“secreted APP” or “sAPP”). Proteases that release APP and its fragments from the membrane are termed “secretases.”

The dominant catabolic pathway appears to be cleavage of APP within the Aβ sequence by α-secretase, resulting in the constitutive secretion of a soluble extracellular domain (sAPPα) and the appearance of a nonamyloidogenic intracellular fragment (approximately 9 kD), referred to as the constitutive carboxy-terminal fragment (cCTFα). cCTFα is a suitable substrate for cleavage by γ-secretase to yield the p3 fragment. This pathway appears to be widely conserved among species and present in many cell types (Weidemann et al., 1989, Cell 57:115-126; Oltersdorf et al., 1990, J. Biol. Chem. 265:4492-4497; and Esch et al., 1990, Science 248:1122-1124). In this pathway, processing of APP involves proteolytic cleavage at a site between residues Lys₁₆ and Leu₁₇ of the Aβ region while APP is still in the trans-Golgi secretory compartment (Kang et al., 1987, Nature 325:773-776). Since this cleavage occurs within the Aβ portion of APP, it precludes the formation of Aβ. sAPPα has neurotrophic and neuroprotective activities (Kuentzel et al., 1993, Biochem. J. 295:367-378).

In contrast to the non-amyloidogenic pathway involving α-secretase, proteolytic processing of APP by β-secretase exposes the N-terminus of Aβ, producing COOH terminal fragments (C-terminal fragments or CTFs) which contain the whole Aβ domain After γ-secretase cleavage at the variable C-terminus, Aβ is liberated. The C-terminus is actually a heterogeneous collection of cleavage sites rather than a single site, since γ-secretase activity occurs over a short stretch of APP amino acids rather than at a single peptide bond. In the amyloidogenic pathway, APP is cleaved by β-secretase to liberate sAPPβ and CTFβ, which CTFβ is then cleaved by γ-secretase to liberate the harmful Aβ peptide.

Of key importance in this Aβ-producing pathway is the position of the γ-secretase cleavage. If the γ-secretase cut is at residue 711-712, short Aβ (Aβ40) is the result; if it is cut after residue 713, long Aβ (Aβ42) is the result. Thus, the γ-secretase process is central to the production of Aβ peptide of 40 or 42 amino acids in length (Aβ40 and Aβ42, respectively). For a review that discusses APP and its processing, see Selkoe, 1998, Trends Cell. Biol. 8:447-453; Selkoe, 1994, Ann. Rev. Cell Biol. 10:373-403. See also, Esch et al., 1994, Science 248:1122.

Cleavage of APP can be detected in a number of convenient manners, including by detecting the polypeptide or peptide fragments produced by proteolysis. Such fragments can be detected by any convenient means, such as by antibody binding. Another convenient method for detecting proteolytic cleavage is through the use of a chromogenic β-secretase substrate whereby cleavage of the substrate releases a chromogen, e.g., a colored or fluorescent product.

Much interest has focused on the possibility of inhibiting the development of amyloid plaques as a means of preventing or ameliorating the symptoms of Alzheimer's disease. To that end, a promising strategy is to inhibit the activity of at least one of β- and γ-secretase, the two enzymes that together are responsible for producing Aβ. This strategy is attractive because, if the formation of amyloid plaques as a result of the deposition of Aβ is a cause of Alzheimer's disease, inhibiting the activity of one or both of the two secretases would intervene in the disease process at an early stage, before late-stage events such as inflammation or apoptosis occur. Such early stage intervention is expected to be particularly beneficial (see, e.g., Citron, 2000, Molecular Medicine Today 6:392-397).

For these reasons, it would be desirable to provide methods and systems for screening test compounds for the ability to inhibit or prevent the production of Aβ from APP, or to inhibit the accumulation of Aβ in the brain. In particular, it would be desirable to base such methods and systems on a metabolic pathway which is involved in such conversion, where the test compound would be able to interrupt or interfere with the metabolic pathway which leads to conversion. In particular, initial methods should utilize in vitro systems rather than animal models, so that the methods are particularly suitable for initial screening of test compounds to become suitable candidate drugs.

### SUMMARY OF THE INVENTION

The invention is directed in part to assays for identifying and evaluating the binding properties of substances to the amyloid precursor protein (APP), or to β-amyloid (Aβ fragments of APP.

In one embodiment, the invention is directed to a process for determining the interaction of a substance to amyloid precursor protein (APP) or amyloid p peptide (Aβ), wherein the method comprises:

(a) forming a first test solution comprising (i) APP or an APP variant having an sAPPβ region and an Aβ region, (ii) a substance, and (iii) a first antibody, wherein the first antibody is specific to the sAPPβ region of the APP or APP variant, under conditions favoring formation of an sAPPβ-antibody complex, to give a first test signaling reading of the sAPPβ-antibody complex;

(b) forming a second test solution comprising (i) the APP or APP variant having an sAPPβ region and an Aβ region, (ii) the substance, and (iii) a second antibody, wherein the second antibody is specific to the Aβ region of the APP or APP variant, under conditions favoring formation of an Aβ-antibody complex, to give a second test signaling reading of the Aβ-antibody complex;

(c) forming a control solution comprising (i) the APP or APP variant having an sAPPβ region and an Aβ region, and (ii) the first and second antibodies, under conditions favoring formation of an sAPPβ-antibody complex and an Aβ-antibody complex, to give a first control signaling reading of an sAPPβ-antibody complex and a second control signaling reading of an Aβ-antibody complex;

(d) comparing the first test signaling reading and the first control signaling reading, and comparing the second test signaling reading and the second control signaling reading,
wherein:
(i) if the first test signaling reading is equal to the first control signaling reading and the second test signaling reading is equal to the second control signaling reading, then the substance does not interact with APP,
(ii) if the first test signaling reading is lower than the first control signaling reading and the second test signaling reading is equal to the second control signaling reading, then the substance binds specifically to the sAPPβ region of APP,
(iii) if the first test signaling reading is equal to the first control signaling reading and the second test signaling reading is lower than the second control signaling reading, then the substance binds specifically to the Aβ region of APP.

In certain embodiments, the antibody specific to Aβ is specific to amino acids 1-17 or 28-40 of Aβ. In other embodiments, the antibody specific to Aβ is specific to the C-terminus of the Aβ region. In other embodiments, the antibody specific to the Aβ region is specific to the N-terminus of the Aβ region.

In another embodiment, the invention is directed to a process for determining the interaction of a substance to amyloid β peptide (Aβ), wherein the method comprises:

(a) forming a first test solution comprising (i) APP or APP variant having an sAPPβ region and an Aβ region, said Aβ region having a first sub-region and a second sub-region, and (ii) a substance, and (iii) a first antibody, wherein the first antibody is specific to the first sub-region of the Aβ region of the APP or APP variant, under conditions favoring formation of an Aβ-antibody complex, to give a first test signaling reading of the Aβ-antibody complex;

(b) forming a second test solution comprising (i) the APP or APP variant having an sAPPβ region and an Aβ region having the first sub-region and the second sub-region, (ii) the substance, and (iii) a second antibody, wherein the second antibody is specific to the first sub-region of the Aβ region of the APP or APP variant, under conditions favoring formation of an Aβ-antibody complex, to give a second test signaling reading of the Aβ-antibody complex;

(c) forming a control solution comprising (i) the APP or APP variant having an sAPPβ region and an Aβ region having the first sub-region and the second sub-region, and (ii) the first and second antibodies, under conditions favoring formation of an Aβ-antibody complex, to give a first control signaling reading of an Aβ-antibody complex and a second control signaling reading of an Aβ-antibody complex;

(d) comparing the first test signaling reading and the first control signaling reading, and comparing the second test signaling reading and the second control signaling reading,
wherein:
(i) if the first test signaling reading is equal to the first control signaling reading and the second test signaling reading is equal to the second control signaling reading, then the substance does not interact with Aβ,
(ii) if the first test signaling reading is lower than the first control signaling reading and the second test signaling reading is equal to the second control signaling reading, then the substance binds specifically to the first sub-region of Aβ,
(iii) if the first test signaling reading is equal to the first control signaling reading and the second test signaling reading is lower than the second control signaling reading, then the substance binds specifically to the second sub-region of Aβ.

In a preferred embodiment of the invention, the first sub-region of Aβ is the C-terminus, and the second sub-region of Aβ is the N-terminus.

In the assays of the invention, the test solutions and the control solutions may further comprise a diluent, a pH control agent, or other additives and inactive carriers commonly used in assays. The test and control solutions may be incubated for up to about 24 hours.

In certain embodiments, the substance has a molecular weight below about 2 kD, preferably in the range of about 100 Da to 1,000 Da.

In certain embodiments, the APP used in the assays is detectably labeled, for example detectably labeled with a ligand.

In certain embodiments, the test and control solutions comprise an acceptor bead.

In another embodiment, the invention is directed to substances that interact with APP or Aβ, which are identified by the assays of the invention. In certain embodiments, the substances are small molecules, with a molecular weight of below about 2 kD. In one embodiment, the substances are benzofuran derivatives selected from the group consisting of compounds of formula I 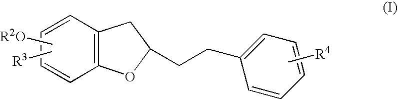
wherein R⁴ is optionally present and is selected from the group consisting of
(a) halogen,
(b) C₁₋₆ alkyl, and
(c) C₁₋₆ alkoxy;
R² is selected from the group consisting of
   (a) hydrogen,
   (b) (CH2)m —C(═O)—OR⁵ wherein R⁵ is selected from the group consisting of hydrogen and C₋₆ alkyl and m is 1, 2 or 3;
R³ is selected from the group consisting of
   (a) hydrogen, and
   (b) 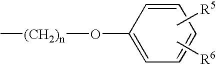
   wherein R⁵ and R⁶ are selected from the group consisting of
      (i) hydrogen,
      (ii) halogen,
      (iii) C₁₋₆ alkyl,
      (iv) C₁₋₆ alkoxy, and
      (v) tetrazolyl, and
      n is 3, 4 or 5,
         or a pharmaceutically acceptable salt thereof. The compounds of formula (I) are capable of interacting with APP and Aβ, and are capable of blocking interaction of APP or Aβ with secretase or APP or Aβ binding antibodies. The compounds of formula I are therefore potentially useful in the treatment of Alzheimer's disease.

The invention is also directed to a method for treating Alzheimer's disease in a patient in need thereof, comprising administering to the patient a therapeutically effective amount off a compound of formula (I). The invention is also directed to methods for ameliorating or controlling Alzheimer's disease in a patient in need thereof, comprising administering to the patient a therapeutically effective amount of a compound of formula (I).

### BRIEF DESCRIPTION OF THE DRAWINGS

FIG. 1 depicts mass spectrometric analysis of Aβ cleavage by BACE2 affected by the compounds of Examples 1 and 4;

FIGS. 2(A) and 2(B) depict Western blot analysis of the inhibition of BACE 1 cleavage of APP by the compound of Example 2;

FIG. 3 depicts the inhibition of BACE1 cleavage of full-length APP or an APP fragment by Statine-Val and the compound of Example 4;

FIG. 4 depicts the reduction of Aβ 40 and 42 levels in H4-C99 cells; and

FIG. 5 depicts a graph of the binding of APP by the compound of Example 1.

### DETAILED DESCRIPTION OF THE INVENTION

Definitions

As used herein, the term “beta-secretase” or “β-secretase” refers to an enzyme that is sometimes known in the literature as “BACE”, “BACE1” (see, e.g., Vassar et al., 1999, Science 286:735-741), or BACE2 (see, e.g., Farzan et al., 2000, PNAS 97:9712-9717). BACE1 is a 501 amino acid membrane-bound aspartic protease. BACE1 has all the known functional properties and characteristics of β-secretase. BACE2, also called Asp-1 or memapsin-1, is a second member of the BACE family of membrane-bound aspartic proteases. See Roggo, Current Topics in Medicinal Chemistry, 2002, 2:359-370, for a further discussion of the differences between BACE1 and BACE2.

As used herein, the term “γsecretase” or “gamma secretase” refers to the gamma secretase enzyme, which is a critical enzyme releasing the Aβ peptide from membrane bound APP processing intermediates. Gamma secretase is believed to be a multi-protein complex, which includes two presenilin fragments, as well as the co-factors nicastrin, Pen-2, and Aph-1. See Schroeter et al, 2003, Proc. Nat'l Acad. Sci., 100:13075-13080; Beher et al, 2003, Biochem 42:8133-8142.

Further, the terms “beta-secretase” and “gamma secretase” as used herein includes all mammalian forms of the naturally occurring enzymes with ability to cleave at the beta-site and gamma site in APP, respectively. The terms “beta-secretase” and “gamma secretase” as used herein also include all recombinant forms, mutations, and other variants of such enzyme so long as these maintain a functional capability to catalyze the cleavage of beta-secretase or gamma secretase substrate at a level of at least about five percent of the effectiveness of a naturally occurring beta-secretase or gamma secretase on the same substrate.

As used herein, the term “variant” denotes in the context of this invention a sequence, whether a nucleic acid or amino acid, that retains a biological activity (either functional or structural) that is substantially similar to that of the original sequence. This variant or equivalent may be from the same or different species and may be a natural variant or be prepared synthetically. Such variants include amino acid sequences having substitutions, deletions, or additions of one or more amino acids, provided the biological activity of the protein is conserved. The same applies to variants of nucleic acid sequences which can have substitutions, deletions, or additions of one or more nucleotides, provided that the biological activity of the sequence is generally maintained.

As used herein, the terms “APP variant” or “variant beta secretase substrate,” are used interchangeably.

An exemplary APP variant (or variant beta-secretase substrate), is a polypeptide molecule that contains any of the novel beta-secretase P2-P1-P1′-P2′ cleavage sites disclosed in International Application No. PCT /US02/15590, published as WO 02/094985, and commonly owned U.S. patent application publication no. US 2003/0200555 A1 (both of which are hereby incorporated by reference), whether or not such molecules also are comprised of additional epitopes to facilitate analysis. For instance, polypeptides that are comprised of 2, 3, 4, 5 or 6 amino acids attached either to one ox both sides of a beta-secretase P2-P1-P1′-P2′ cleavage site are modified beta-secretase substrates. Modified beta-secretase substrates include non-naturally occurring peptides, each of which comprises a contiguous sequence fragment of at least 8 amino acids, such fragment comprising a synthetic beta-secretase cleavage site, where the at least 8 amino acids have a sequence selected from the group of sequences disclosed in WO 02/094985 and US 2003/0200555 A1. As similarly noted therein, fragments and homologs of such non-naturally occurring peptides also are encompassed by the present invention. Full-length APP molecules, whether of 695, 751 or 770 amino acid lengths, and such molecules with additional amino acids added to either or both ends, are modified beta-secretase substrates so long as they possess a beta-secretase P2-P1-P1′-P2′ cleavage site, as described above. Also, intermediate-sized polypeptides smaller than the full-length APP molecules of 695, 751 or 770 amino acid lengths but larger than the aforementioned multimers up to 16 polypeptides in length, which are substantially comprised of the APP sequence and which are cleavable by beta-secretase, are modified beta-secretase substrates so long as they possess a beta-secretase P2-P1-P1′-P2′ cleavage site, as described above. Such intermediate-sized polypeptides are also referred to as “biologically active fragments” or “biologically active amino acid fragments” of APP, “APP biologically active fragments,” or “APP biologically active amino acid fragments.” Reference to such intermediate-sized polypeptide is limited to these terms, to avoid confusion with the use of the term “fragment” when referring, infra, to peptides and polypeptides that result from enzymatic cleavage by beta-secretase alone or in conjunction with other enzymes, such as gamma-secretase.

The term “derivative” is intended to include any of the above described variants when comprising additional chemical moieties not normally a part of these molecules. These chemical moieties can have varying purposes including, improving a molecule's solubility, absorption, biological half life, decreasing toxicity and eliminating or decreasing undesirable side effects. Furthermore, these moieties can be used for the purpose of labeling, binding, or they may be comprised in fusion product(s). Different moieties capable of mediating the above described effects can be found in Remington's The Science and Practice of Pharmacy (1995). Methodologies for coupling such moieties to a molecule are well known in the art.

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid); substitution of one aromatic amino acid (tryptophan, tyrosine, or phenylalanine) for another.

A “conservative amino acid substitution” as defined above is but one type of variation of an amino acid sequence listing encompassed by the broader term, “conservatively modified variants thereof.” For instance, the latter is taken to have the meaning ascribed to the term in M.P.E.P. § 2422.03, Eighth Edition, 2001, which can include, without being limited to this example, deletions such as “at the C-terminus by 1, 2, 3, 4, or 5 residues.”

For instance, but not meant to be limiting, an amino acid sequence or a nucleotide sequence is considered “identical” to a reference sequence if the two sequences are the same when aligned for maximum correspondence over a comparison window. Optimal alignment of nucleotide and amino acid sequences for aligning a comparison window may be conducted by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Natl. Acad. Sci., U.S.A. 85:2444-2448, by computerized implementations of these algorithms (GAP, BESFIT, FASTA, and TFASTA in the Wisconsin Genetics Software Package Release 7.0, Genetics Computer Group, 575 Science Dr., Madison, Wis.), or by inspection. Such determination of identity can be considered to indicate a “conservatively modified variant” of a particular amino acid sequence or nucleotide sequence so long as the variant continues to function as a beta-secretase substrate.

In the alternative, comparative similarity of a variant, for purposes of the invention, is defined by the “relative sequence identity” of that variant to a novel protein or polypeptide sequence, so long as the comparative similarity falls within a specified boundary and the variant continues to be a substrate for the beta-secretase enzyme. For instance, a particular APP backbone (or polypeptide regions thereof) can have any or all of the following: substitutions, deletions, insertions, and additions. Certain approaches and specific methods for making variants with substitutions, deletions, insertions, and/or additions, are within the knowledge of those skilled in the art. Thus, in certain embodiments, a protein or polypeptide variant of a specified claimed sequence number that is at least 95 percent identical to that sequence number (based on amino acid sequence homology, i.e., the “relative sequence identity”), where that variant is demonstrated to be a substrate for the beta-secretase enzyme, is within the scope of the present invention. In other embodiments, a protein or polypeptide variant of a specified claimed sequence number that is at least 85 percent identical to that sequence number (based on amino acid sequence homology), where that variant is demonstrated to be a substrate for the beta-secretase enzyme, is within the scope of the present invention. In yet other embodiments, a protein or polypeptide variant of a specified claimed sequence number that is at least 65 percent identical to that sequence number (based on amino sequence homology), where that variant is demonstrated to be a substrate for the beta-secretase enzyme, is within the scope of the present invention. For all such variants, it is noted that the functional ability to serve as a “suitable substrate” for the beta-secretase enzyme, such as in test systems described herein, or also in other methods now known in the art, or, optionally, also including methods later known in the art, is essential to the inclusion of any such variant within the operation of the invention, and the scope of any relevant claim.

“Aβ variant peptides” are amyloid peptides which are variants, as that term is defined above, of the amyloid β peptide (Aβ).

“Consists essentially” with respect to a beta-secretase substrate, indicates that the reference sequence can be modified by N-terminal and/or C-terminal additions or deletions that do not cause a substantial decrease in the ability of the beta-secretase substrate to be cleaved compared to the reference sequence. An example of a deletion is the removal of an N-terminal methionine.

The term “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Antibodies exist as intact immunoglobulins or as a number of well-characterized fragments produced by digestion with various peptidases. These include, e.g., Fab′ and F(ab)′₂ fragments. The term “antibody” also includes antibody fragments either produced by the modification of whole antibodies or those synthesized de novo using recombinant DNA methodologies, and further includes “humanized” antibodies made by conventional techniques.

The term “immunoassay” is an assay that uses an antibody to specifically bind an analyte. The immunoassay is characterized by the use of specific binding properties of a particular antibody to isolate, target, and/or quantify the analyte.

An antibody “specifically binds to” or “is specifically immunoreactive with” a protein, polypeptide, or peptide when the antibody functions in a binding reaction which is determinative of the presence of the protein, polypeptide, or peptide in the presence of a heterogeneous population of proteins and other biologics. Thus, under designated immunoassay conditions, the specified antibodies bind preferentially to a particular protein, polypeptide, or peptide and do not bind in a significant amount to other proteins, polypeptides, or peptides present in the sample. Specific binding to a protein, polypeptide, or peptide under such conditions requires an antibody that is selected for specificity for a particular protein, polypeptide, or peptide. As used herein, the term “recognize” as it regards an antibody's association to a particular protein, polypeptide, or peptide, or a region or epitope therein, is taken to mean that said antibody “specifically binds to” or “is specifically immunoreactive with” that protein, polypeptide, or peptide, or region or epitope therein.

Antibodies suitable for use in the invention include any antibodies which are specific to APP or Aβ, or more accurately are specific to various particular regions of APP or Aβ. Suitable antibodies are described in the literature, and are known to those of ordinary skill in the art. Antobodies specific to particular regions of APP or Aβ may be developed by those of ordinary skill in the art, using conventional techniques. One type of antibody suitable for use in the assays of the invention are antibodies specific to the sAPPβ region of APP. Exemplary sAPPβ specific antibodies include LN27 (which is specific to the N-terminal 200 amino acids of APP), available from Zymed Laboratories of South San Francisco, Calif.; P2-1, available from Affinity BioReagents of Golden, Colo.; 22C11, available from Boehringer Mannheim of Indianapolis, Ind., and S-12, available from Alpha Diagnostic International, Inc. of San Antonio, Tex.

Another type of antibody suitable for use in the assays of the invention are antibodies specific to the Aβ region of APP. Exemplary antibodies specific to the Aβ region include WO-2, available from Toray Company, Tokyo, Japan; and 6E-10, available from Signet Laboratories of Dedham, Mass.

Another type of antibody suitable for use in the assays of the invention are antibodies specific to the C-terminus of Aβ. Exemplary Aβ C-terminus antibodies include G2-10, which is available from Toray Company of Tokyo, Japan.

Another type of antibody suitable for use in the assays of the invention are antibodies specific to the N-terminus of Aβ.

The protein, polypeptide, or peptide that combines specifically with antibodies, and the processed peptides that combine specifically with cell-surface receptors of immune cells, are referred to as “antigens.”

An “immunogenic peptide” is defined as a peptide sequence that is of sufficient length and amino acid composition to induce an immune response (humoral and/or cell-mediated) in a suitable host animal when injected therein with a suitable range of concentrations. An immunogenic peptide that includes all or part of a modified beta-secretase cleavage site (such as, for example, the modified beta-secretase cleavage sites disclosed in WO 02/094985 and US 2003/0200555 A1) results in production in the host animal of an antibody which ultimately recognizes the peptide sequence of the modified beta-secretase cleavage site as a free peptide and also when at an end of the beta-secretase processed APP and APP variants. The aforementioned immunogenic peptides also possess the property of antigens, i.e., such immunogenic peptides are antigens in that each such immunogenic peptide combines specifically with particular antibodies raised against such specific peptide.

A variety of immunoassay formats may be used to select antibodies specifically immunoreactive with a particular protein, polypeptide, or peptide. For example, solid-phase ELISA immunoassays are routinely used to select monoclonal antibodies specifically immunoreactive with a protein, polypeptide, or peptide. See Harlow & Lane, 1988, Antibodies, A Laboratory Manual, Cold Spring Harbor Publications, N.Y., for a description of immunoassay formats and conditions that can be used to determine specific immunoreactivity.

As used herein, the term “substance” means any molecule, compound, mixture of molecules or compounds, or any other composition which is suspected of interacting with APP or Aβ, and capable of blocking interaction of APP or Aβ with secretases or APP or Aβ binding antibodies. “Substances” that are screened in the present invention can be any substances that are generally screened in the pharmaceutical industry during the drug development process. The substances may be macromolecules, such as biological polymers, including proteins, polysaccharides, nucleic acids, or the like. More usually, a substance will be a compound or small molecule having a molecular weight below about 2 kD, more usually below 1.5 kD, frequently below 1 kD, and usually in the range from 100 Da to 1,000 Da, and even more usually in the range from 200 Da to 750 Da. One or more substances may be pre-selected based on a variety of criteria. For example, suitable substances may be selected as having known proteolytic inhibitory activity. Alternatively, the substances may be selected randomly and tested by the screening methods of the present invention. Substances which are able to block interaction of APP or Aβ with secretases or APP or Aβ binding antibodies in vitro are considered as candidates for further screening of their ability to decrease Aβ production in cells and/or animals. Substances may be tested in the methods of the present invention as large collections of substances, e.g. libraries of low molecular weight organic compounds, peptides, or natural products.

While the assays and screening methods described herein are explicitly directed to testing “a” substance, it will be clear to a person skilled in the art that such a method can be adapted to testing multiple substances, e.g., combinatorial libraries to determine if any member of such a collection binds APP. Accordingly, the use of collections of substances, or individual members of such collections is within the scope of this invention.

As used herein, the term “assay conditions” are conditions which are conducive to the formation of a bonding relationship desired in the assay. For example, assay conditions may be conditions which are conducive to or favor formation of a complex between an antibody and an antigen (e.g., conditions favoring formation of an sAPPβ-antibody complex). The assay conditions may include use of a pH control agent, for example sodium acetate; a diluent, for example DMSO; a buffer, for example a buffering solution comprising PIPES; a detergent, for example CHAPS; and/or a blocking agent, for example BSA. Assay conditions may also include use of a commercially available assay cocktail, for example a commercially available protease inhibitor cocktail.

As used herein, the term “acceptor beads” refers to inert beads commonly used in assays. Exemplary acceptor beads include acceptor beads from AlphaScreen General IgG detection kit, available from Perkin Elmer Life Sciences.

The term “fragment” refers to any segment of an identified amino acid sequence and/or any segment of any of the variants or derivatives described herein above.

“sAPP beta fragment” or “sAPPβ” refers to an approximately 100 kD amino terminal fragment produced when APP is cleaved by beta-secretase.

“C-terminal fragment” or “CTFβ” refers to an approximately 12 kD 99 amino acid (also known as CTF99) COOH-terminal fragment produced when APP is cleaved by beta-secretase. Alternatively, “C-terminal fragment” or “CTFβ” may refer to an 100 amino acid engineered protein (also known as CTF100 or C100) containing the 99 C-terminal amino acids of APP and an N-terminal methionine. CTFβ contains the entire Aβ domain.

The β-secretase assays described herein are conveniently performed using “sandwich” assays where the amino-terminal or the carboxy-terminal fragment produced by cleavage is captured on a solid phase. The captured fragment may then be detected using an antibody specific for the end of the fragment exposed by β-secretase cleavage. Exemplary antibodies include antibodies raised against any cleavage products produced as a result of β-secretase activity. The binding of the antibody to the cleaved cleavage product is detected using conventional labeling systems, such as horseradish peroxidase or other detectable enzyme labels, which are bound to the antibody directly (covalently), or indirectly through intermediate linking substances, such as biotin and avidin. Such “sandwich” assays can be performed in various formats, including IGEN based technology, HTRF, Alpha Screen technology, and other technologies known to those of ordinary skill in the art.

The term “measurable signal” as used herein includes any type of generated signal, e.g., radioactive, calorimetric, photometric, spectrophotometric, scintillation, which is provided upon binding of the radioligand binding agent to the target.

As used herein, the term “C-terminus” or “carboxy-terminus” is the region of a protein or peptide containing the terminal a-carboxyl group. The terms are used herein to refer to the region containing the terminal a-carboxyl group of the amyloid β peptide.

As used herein, the term “N-terminus” or “amino terminus” is the region of a protein or peptide containing the terminal a-amino group. The terms are used herein to refer to the region containing the terminal a-amino group of the amyloid 13 peptide.

As used herein, the term “secretase” refers to a protease that cleaves APP to form amyloidogenic peptides, including BACE1, BACE2 and gamma secretase.

As used herein, the term “vehicle” refers to an inactive medium for a carrier or active substance used as a control in an experiment. For example, as used in an assay of the invention as described herein, a substance of interest is incubated, usually with a diluent, pH control agent, and other inactive carriers commonly used by those of ordinary skill in the art to conduct assays. In the “vehicle” used as a control in the assay, the same diluent, pH control agent, and other inactive carriers are present.

As used herein, the term “interaction” refers to the properties of binding or chemical attraction between a substance of interest, ligand or antibody and a biological receptor. “Interaction” as used herein refers to any kind of chemical bonding between the substance ligand or antibody and the receptor, including covalent bonds, hydrogen bonds or ionic bonds.

In one embodiment, the invention is directed to an assay for detecting and evaluating the interaction of a substance with the sAPPβ region of APP, and for detecting and evaluating the interaction of a substance with Aβ.

In this embodiment, the full APP protein (or APP variant protein) is incubated in a first test solution with the substance of interest and with a first antibody, which is specific for the sAPPβ region of APP. A second test solution is formed, comprising a second antibody, which is specific for the Aβ region of APP, and the substance of interest and APP or APP variant. The second antibody may be specific to various regions of Aβ. For example the second antibody may be specific to amino acids 1-17 or 28-40 of Aβ. Alternatively, the second antibody may be specific to the C-terminus or N-terminus of Aβ.

Two control solutions are formed under assay conditions, comprising vehicle, the first antibody and APP or variant, and vehicle, the second antibody, and the APP or variant. A third and fourth control solution, comprising respectively the first antibody and the substance of interest, and the second antibody and the substance of interest, may also be formed.

The binding properties of each of the first and second antibodies, in each of the first and second test solutions and first and second control soutions, is measured. The binding measurements indicate the extent to which the substance of interest interacts with or binds to the sAPPβ region of APP and to Aβ.

In order to determine the binding properties of the substance of interest, the APP or variant may be detectably labeled with one ligand of a binding pair of ligands. A suitable labeled APP or variant is biotinylated APP or variant.

In certain embodiments, the APP is an APP variant which is the modified beta-secretase substrate that contains any of the novel beta-secretase P2-P1-P1′-P2′ cleavage sites disclosed in WO 02/094985 and US 2003/0200555 A1.

The substance and APP (or variant) may be incubated at about 37° C., for up to about 24 hours, preferably for about one hour. A solution of the incubated mixture may then be placed in one well of a multi-well plate. A stabilizing agent, for example 1M Tris-Cl, pH 8.0, may also be added to the mixture to neutralize binding reactions.

In one embodiment, beads for use in the assay are prepared. For example, a first bead mixture is formed of (1) donor beads comprising streptavadin, (2) acceptor beads (e.g., Protein A acceptor beads from AlphaScreen General IgG Detection Kit Perkin Elmer Life Sciences) containing polyclonal antibody directed against amino acids 28-40 of Aβ, (3) BSA, and (4) phosphate buffered saline, and a second bead mixture is formed of (1) donor beads comprising streptavadin, (2) acceptor beads (e.g., Protein A acceptor beads from AlphaScreen General IgG Detection Kit Perkin Elmer Life Sciences) containing monoclonal mouse antibody directed against the N-terminal 200 amino acids of APP, (3) BSA, and (4) phosphate buffered saline.

Thereafter, the first bead mixture is combined with an incubated mixture of the substance of interest and APP (or variant), and the mixture is incubated overnight. The second bead mixture is combined with an incubated mixture of the substance of interest and APP (or the variant). Third and fourth mixtures, comprising vehicle, the APP or APP variant, and acceptor beads from the first and second mixtures described above, respectively, are formed. The well-plates are then read.

The streptavidin-coated donor beads interact with the biotin moiety present on APP (or variant), and the acceptor beads bind to APP (or variant) through interactions with the antibody which binds to either the N-terminal or the Aβ domain of APP. Substances that do not interact with APP at either the first or second antibody binding sites produce a signal which is approximately the same as vehicle. Substances which interact non-specifically with APP fall out of solution, since the APP is not accessible to any antibody, and the signal is reduced relative to vehicle when either the anti-Aβ or APP N-terminal antibody mixture are used. Substances which interact specifically with APP near the binding site of one of the first or second antibodies produce a signal which is reduced specifically for the antibody proximal to the substance binding site, but not reduced for the more distal antibody.

In a second embodiment, the invention is directed to an assay for detecting and evaluating the interaction of a substance with the N-terminus and C-terminus of Aβ or Aβ variant peptides. Measurement of the binding properties of the substance can determine the extent to which the substance interacts with or binds to Aβ or Aβ variant peptides.

In this embodiment, Aβ (or variant) is mixed with a buffer, for example BSA, in a phosphate buffered saline, under assay conditions. A control mix of binding buffer is also formed. A solution of the binding mix is then placed in one well of a multi-well plate. The binding reaction is then incubated at about 37° C. for about one hour. A solution of each reaction is transferred to a second assay plate, where the interaction between Aβ (or an Aβ variant peptide) and an antibody is assessed.

A first bead mixture may be formed of (1) donor beads comprising streptavadin, and (2) a labelled antibody which is specific for the C-terminus of Aβ. A second bead mixture may be formed of (1) acceptor beads (e.g., Protein A acceptor beads from AlphaScreen General IgG Detection Kit Perkin Elmer Life Sciences), and (2) an antibody which is specific to the amino terminus of Aβ.

The mixtures are incubated at room temperature for about one hour prior to the addition of the amyloid binding reaction.

In each well of a multi-well plate, the first bead mixture, the second bead mixture, and a buffer (e.g., BSA in PBS) are added. The plates are then incubated overnight, and antibody binding to the N-terminus of Aβ is detected (e.g., via ALPHAQUEST). A reduction in signal is demonstrated if the reaction is carried out in the presence of a substance that interacts with Aβ.

The assays as described above may use the AlphaScreen™ technology developed by BioSignal Packard, Inc., Meriden, Conn. Other binding assay technologies exist in the art in which the basic principles of this invention can be used, namely, contacting a mixture of APP and a substance with a first antibody, wherein the first antibody is specific to the sAPP region of APP, and a second antibody, wherein the second antibody is specific to the Aβ region of APP, and determining the binding properties of the substance. These other binding assays include ligand-binding assays, for example, calorimetric and spectrophotometric assays, e.g. measurement of produced color or fluorescence, phosphorescence (e.g. ELISA, solid absorbant assays) and other radioimmunoassays in which short or long wave light radiation is produced (including ultraviolet and gamma radiation).

In a third embodiment, the invention is directed to compounds of formula (I), which were identified from the assays of the invention. The compounds of formula (I) interact specifically with APP or Aβ, and block interaction of APP with secretases or with Aβ binding antibodies. The compounds of formula (I) inhibit APP processing by BACE1 and BACE2, inhibit Aβ processing by BACE2, and inhibit CTFβ (including CTF99and CTF100) processing by gamma secretase. It is believed that the compounds of formula (I) inhibit BACE and gamma secretase processing by binding APP within the Aβ domain of APP.

The compounds of the invention may also act as modulators of the action of gamma secretase, so as to selectively attenuate the production of Aβ 1-42, resulting in a preferential secretion of the shorter chain isoforms of Aβ (for example, Aβ40). The shorter chain isoforms are believed to have a reduced propensity for self-aggregation and plaque formation, and hence are more easily cleared from the brain and are less neurotoxic.

Compounds of formula I may be formed by the scheme below.

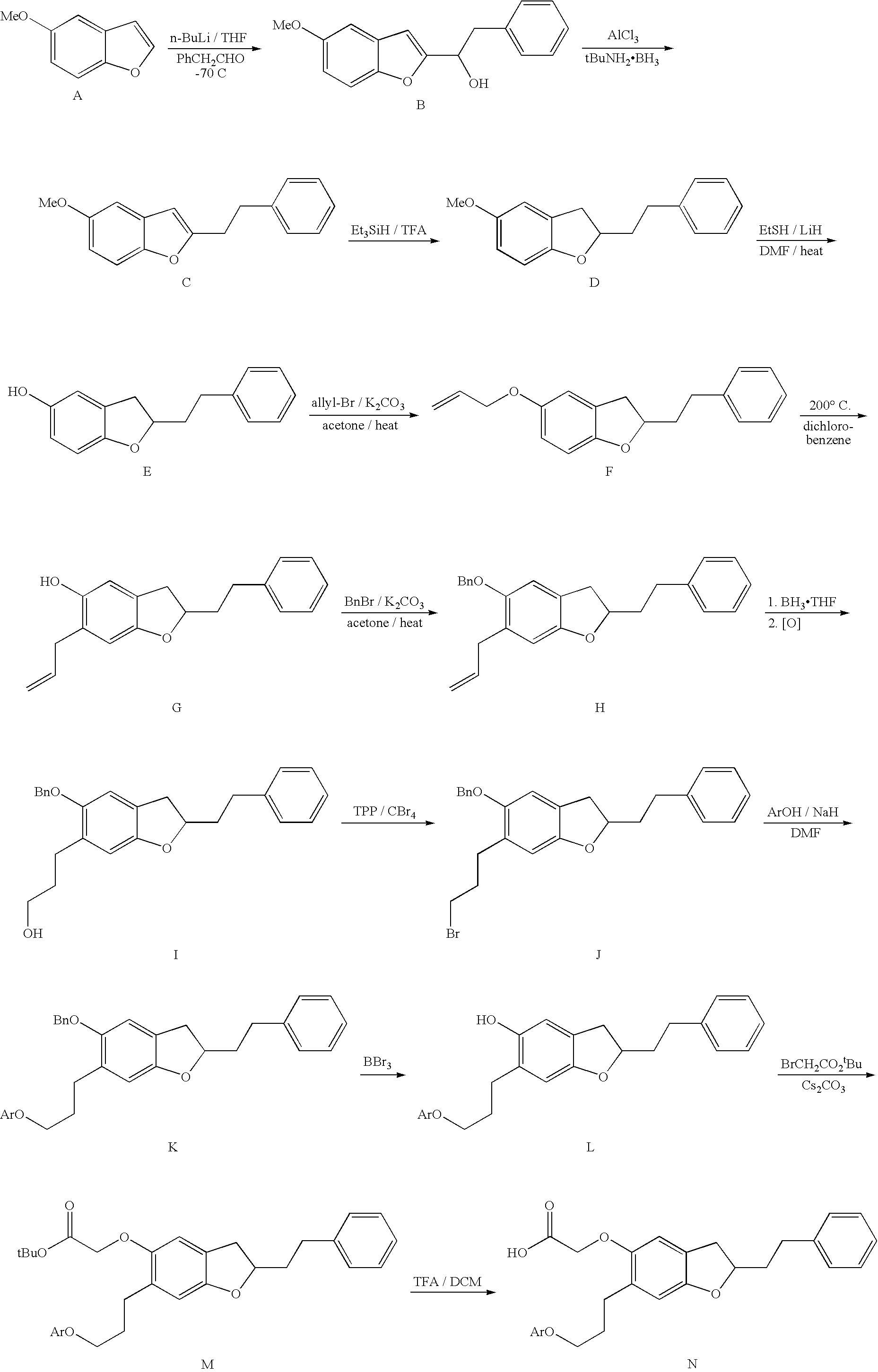

The starting materials and reagents for the processes described herein are either commercially available or are known in the literature or may be prepared following literature methods described for analogous compounds. The skills required in carrying out the reaction and purification of the resulting reaction products are known to those in the art. Purification procedures include crystallization, distillation, normal phase or reverse phase chromatography.

The present invention is further directed to a method for the manufacture of a medicament or a composition for treating Alzheimer's Disease in humans and animals, comprising combining a compound of formula (I) of the present invention with a pharmaceutically acceptable carrier or diluent. The present invention is also directed to a method for the manufacture of a medicament which comprises a compound which interacts specifically with APP or Aβ, and blocks interaction of APP or Aβ with secretases or with APP or Aβ binding antibodies.

Another embodiment of the present invention is to the compounds of formula (I), which are selected from the title compounds of the following Examples 1-4 and pharmaceutically acceptable salts thereof.

The compounds of formula (I) of the present invention have utility in treating, ameliorating, controlling or reducing the risk of Alzheimer's disease. For example, the compounds may be useful for the prevention of dementia of the Alzheimer's type, as well as for the treatment of early stage, intermediate stage or late stage dementia of the Alzheimer's type. The compounds may also be useful in treating, ameliorating, controlling or reducing the risk of diseases mediated by abnormal cleavage of APP, and other conditions that may be treated or prevented by inhibition of BACE cleavage of APP and inhibition of aggregation of Aβ. Such conditions include mild cognitive impairment, Trisomy 21 (Down Syndrome), cerebral amyloid angiopathy, degenerative dementia, Hereditary Cerebral Hemorrhage with Amyloidosis of the Dutch-Type (HCHWA-D), Creutzfeld-Jakob disease, prion disorders, amyotrophic lateral sclerosis, progressive supranuclear palsy, head trauma, stroke, Down syndrome, pancreatitis, inclusion body myositis, other peripheral amyloidoses, diabetes and atherosclerosis.

The compounds of formula (I) of the present invention may also be useful as positron emission tomography (PET) ligands for the detection or diagnosis of AD.

Compounds of the invention may be labeled with positron emitting radionuclides, for us in PET imaging as PET ligands. For imaging, suitable PET radionuclides include ³H, ¹¹C, ¹⁸F, ¹²⁵I, ⁸²Br, ¹²³I, ¹³¹I, ⁷⁵Br, ¹⁵O, ¹³N, ²¹¹At and ⁷⁷Br. The most commonly used PET radionuclides are ¹¹C, ¹⁸F, ¹⁵O and ¹³N. The particular radionuclide that is incorporated in the instant radiolabeled compounds will depend on the specific analytical or pharmaceutical application desired.

The present invention is also directed to a radiopharmaceutical composition which comprises a compound of the present invention and at least one pharmaceutically acceptable carrier or excipient. The present invention is also directed to a method for labeling APP or Aβ in a mammal, which comprises administering to a mammal in need of such labeling an effective amount of the radiolabeled compound of the present invention.

The present invention is also directed to a method for diagnostic imaging of APP or Aβ in a mammal which comprises administering to a mammal in need of such diagnostic imaging an effective amount of the radiolabeled compound of the present invention.

The present invention is also directed to a method for diagnostic imaging of the brain in a mammal which comprises administering to a mammal in need of such diagnostic imaging an effective amount of the radiolabeled compound of the present invention.

In a preferred embodiment of the methods of the present invention, the mammal is a human.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amine s, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of formula (I) of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids.

As used herein, the term “alkyl,” by itself or as part of another substituent, means a saturated straight or branched chain hydrocarbon radical having the number of carbon atoms designated (e.g., C₁₋₁₀ alkyl means an alkyl group having from one to ten carbon atoms). Preferred alkyl groups for use in the invention are C₁₋₆ alkyl groups, having from one to six carbon atoms. Exemplary alkyl groups include methyl, ethyl, n-propyl, isopropyl, n-butyl, isobutyl, tert-butyl, pentyl, hexyl, and the like.

As used herein, the term “alkoxy,” by itself or as part of another substituent, means a group —O— R, wherein R is an alkyl group as defined above. Preferred alkoxy groups for use in the invention are C₁₋₆ alkoxy groups, having from one to six carbon atoms. Exemplary alkoxy groups include methoxy, ethoxy, n-propoxy, isopropoxy, and the like.

The term “halo” or “halogen” includes fluoro, chloro, bromo and iodo.

The subject or patient to whom the compounds of formula (I) of the present invention is administered is generally a human being, male or female, in whom specific interaction with APP or Aβ is desired, but may also encompass other mammals, such as dogs, cats, mice, rats, cattle, horses, sheep, rabbits, monkeys, chimpanzees or other apes or primates, for which specific interaction with APP or Aβ, or with APP or Aβ binding antibodies, is desired.

The compounds of formula (I) of the present invention may be used in combination with one or more other drugs in the treatment of diseases or conditions for which the compounds of formula (I) of the present invention have utility, where the combination of the drugs together are safer or more effective than either drug alone. Additionally, the compounds of formula (I) of the present invention may be used in combination with one or more other drugs that treat, prevent, control, ameliorate, or reduce the risk of side effects or toxicity of the compounds of formula (I) of the present invention. Such other drugs may be administered, by a route and in an amount commonly used therefore, contemporaneously or sequentially with the compounds of formula (I) of the present invention. Accordingly, the pharmaceutical compositions of the present invention include those that contain one or more other active ingredients, in addition to the compounds of formula (I) of the present invention. The combinations may be administered as part of a unit dosage form combination product, or as a kit or treatment protocol wherein one or more additional drugs are administered in separate dosage forms as part of a treatment regimen.

Examples of combinations of the compounds of formula (I) of the present invention with other drugs in either unit dose or kit form include combinations with one or more of: anti-Alzheimer's agents, for example other beta-secretase inhibitors or gamma-secretase inhibitors; HMG-CoA reductase inhibitors; NSAID's including ibuprofen; vitamin E; anti-amyloid antibodies, including humanized monoclonal antibodies; cathepsin B inhibitors; CB-1 receptor antagonists or CB-1 receptor inverse agonists; antibiotics such as doxycycline and rifampin; N-methyl-D-aspartate (NMDA) receptor antagonists, such as memantine; cholinesterase inhibitors such as galantamine, rivastigmine, donepezil, and tacrine; growth hormone secretagogues such as ibutamoren, ibutamoren mesylate, and capromorelin; histamine H₃ antagonists; AMPA agonists; PDE IV inhibitors; GABA_{A} inverse agonists; neuronal nicotinic agonists; or other drugs that affect receptors or enzymes that either increase the efficacy, safety, convenience, or reduce unwanted side effects or toxicity of the compounds of formula (I) of the present invention. The foregoing list of combinations is illustrative only and not intended to be limiting in any way.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Other pharmaceutical compositions include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the invention may also be in the form of oil-in-water emulsions, which may also contain excipients such as sweetening and flavoring agents.

The pharmaceutical compositions may be in the form of a sterile injectable aqueous or oleaginous suspension, which may be formulated according to the known art, or may be administered in the form of suppositories for rectal administration of the drug.

The compounds of formula (I) of the present invention may also be administered by inhalation, by way of inhalation devices known to those skilled in the art, or by a transdermal patch.

By “pharmaceutically acceptable” it is meant the carrier, diluent or excipient must be compatible with the other ingredients of the formulation and not deleterious to the recipient thereof.

The terms “administration of” or “administering a” compound should be understood to mean providing a compound of formula (I) of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” means the amount of the subject compound that will elicit the biological or medical response of a tissue, system, animal or human that is being sought by the researcher, veterinarian, medical doctor or other clinician. As used herein, the term “treatment” refers to the treatment of the mentioned conditions, particularly in a patient who demonstrates symptoms of the disease or disorder.

As used herein, the term “treatment” or “treating” means any administration of a compound of the present invention and includes (1) inhibiting the disease in an animal that is experiencing or displaying the pathology or symptomatology of the disease (i.e., arresting further development of the pathology and/or symptomatology), or (2) ameliorating the disease in an animal that is experiencing or displaying the pathology or symptomatology of the disease (i.e., reversing the pathology and/or symptomatology). The term “controlling” includes preventing treating, eradicating, ameliorating or otherwise reducing the severity of the condition being controlled.

The compositions containing compounds of formula (D of the present invention may conveniently be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples of unit dosage forms.

The compositions containing compounds of formula (V) of the present invention may conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

When treating, ameliorating, controlling or reducing the risk of Alzheimer's disease or other diseases for which compounds of formula (I) of the present invention are indicated, generally satisfactory results are obtained when the compounds of formula (I) of the present invention are administered at a daily dosage of from about 0.1 mg to about 100 mg per kg of animal body weight, preferably given as a single daily dose or in divided doses two to six times a day, or in sustained release form. The total daily dosage is from about 1.0 mg to about 2000 mg, preferably from about 0.1 mg to about 20 mg per kg of body weight. In the case of a 70 kg adult human, the total daily dose will generally be from about 7 mg to about 1,400 mg. This dosage regimen may be adjusted to provide the optimal therapeutic response. The compounds of formula (I) of the present invention may be administered on a regimen of 1 to 4 times per day, preferably once or twice per day.

Specific dosages of the compounds of formula (I) of the present invention, or pharmaceutically acceptable salts thereof, for administration include 1 mg, 5 mg, 10 mg, 30 mg, 80 mg, 100 mg, 150 mg, 300 mg and 500 mg. Pharmaceutical compositions of the present invention may be provided in a formulation comprising about 0.5 mg to 1000 mg active ingredient; more preferably comprising about 0.5 mg to 500 mg active ingredient; or 0.5 mg to 250 mg active ingredient; or 1 mg to 100 mg active ingredient. Specific pharmaceutical compositions useful for treatment may comprise about 1 mg, 5 mg, 10 mg, 30 mg, 80 mg, 100 mg, 150 mg, 300 mg and 500 mg of active ingredient.

It will be understood, however, that the specific do se level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The following examples are provided for the purpose of further illustration only and are not intended to be limitations on the disclosed invention.

### EXAMPLE 1

### {[6-(3-phenoxypropyl)-2-(2-phenylethyl)-2,3-dihydro-1-benzofuran-5-yl]oxy } acetic acid

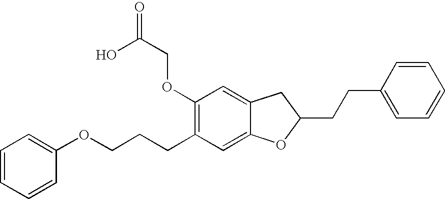

Step A To a solution of 5-methoxybenzofuran (148 gm; 1 mole) in THF (2 L) at −78° C. was added drop-wise 2.5 molar n-butyllithium in hexane (420 mL; 1.05 moles). The mixture was stirred at −78° C. for 1.5 hours and then phenyl acetaldehyde (144 g, 1.2 mole) was added over 15 minutes. The cooling bath was removed and the mixture permitted to gradually rise to 0° C. Water (600 mL) was added. The ether layer was separated, dried over MgSO₄, filtered and concentrated in vacuo. The crude was chromatographed in silica gel (2 kg) using a solvent mixture of increasing polarity of 5%, 10%, 20% and 30% ethyl acetate in hexane to obtain 2-(1-hydroxy-2-phenylethyl)-5-methoxybenzofuran, m.p. 69-70° C.

Step B To a mixture of aluminium chloride (204 gm; 1.5 moles) in tolueme (2.5 L) at 5° C. and under a nitrogen atmosphere was added in 50 gm portions t-butylamine borane (200 gm; 2.3 moles). After stirring for 30 minutes, a solution of compound 1-A (238 g, 880 mmol) in toluene (600 mL) was added drop-wise. The mixture was stirred at 5° C. for 2 hours and then added in portions to a stirring ice cold mixture of 10% hydrochloric acid (3 L). Stirring was continued until fizzing had stopped. The organic layer was separated and dried over MgSO₄, filtered and concentrated in vacuo. The residue was chromatographed in silica gel (2 kg) using 15% ethyl acetate as eluent to obtain 2-(2-phenylethyl)-5-methoxybenzofuran.

Step C Trifluoroacetic acid (232 mL; 30 moles) was added over 15 minutes to a suspension of 2-(2-phenylethyl)-5-methoxybenzofuran from step 1-B (128 gm; 500 mmol) in triethylsilane (465 mL; 2.8 moles) at 5° C. The mixture was stirred at this temperature for 1 hour and at room temperature for 18 hours. The mixture was concentrated and dissolved in ether, washed with 1 N sodium hydroxide, dried over MgSO4, filtered and concentrated. The residue was chromatographed (5% EtOAc/hexanes) to obtain 2-(2-phenylethyl)-5-methoxy-2,3-methoxybenzofuran.

Step D Ethanethiol (62 gm; 1 mole) was added drop-wise to lithium hydride (8 g; 1.0 mol) in DMF (700 mL) under nitrogen atmosphere. 2-(2-phenylethyl)-5-methoxy-2,3-dihydrobenzofuran (126 g, 500 mmol) in DMF (200 mL) was then added in one portion and the mixture brought to reflux for 3 hours. The mixture was poured into 1N hydrochloric acid and extracted with diethyl ether. The ether layer was separated and washed with water twice, dried over MgSO₄, filtered and concentrated in vacuo to obtain 123 g (98%) of 5-hydroxy-2-(2-phenyl-ethyl)-2,3-dihydrobenzofuran, m.p. 56-58° C.

Step E A mixture of 5-hydroxy-2-(2-phenylethyl)-2,3-dihydrobenzoruan (5.0 g, 20.9 mmol), potassium carbonate (5.5 g; 40.0 mmol), allyl bromide (4.8 g, 40.0 mmol) and acetone (50 mL) was refluxed for 18 hours. The mixture was cooled, diluted with hexane (25 mL) and filtered through Celite. The filtrate was concentrated in vacuo and the residue chromatographed on silica gel using 10% EtOAc/hexanes to obtain 5-allyloxy-2-(2-phenylethyl)-2,3-dihydrobenzofuran.

Step F A mixture of 5-allyloxy-2-(2-phenylethyl)-2 3-dihydrobenzofuran (4.7 g, 16.7 mmol) in 1,2-dichlorobenzene (10 mL) was refluxed overnight. The mixture was concentrated and the residue was chromatographed on silica gel using 10% EtOAc/Hexanes to obtain 4.1 g (87%) of pure isomer mixture that was further separated by chromatography (50% Hexanes/DCM) to obtain 6-allyl-5-hydroxy-2-(2-phenylethyl)-2,3-dihydrobenzofuran, m.p. 79-80° C.

Step G A mixture of 6-allyl-5-hydroxy-2-(2-phenylethyl)-2,3-dihydrobenzofuran (2.8 g, 10.0 mmol), potassium carbonate (1.38 g, 10.0 mmol), benzyl bromide (1.7 g, 10.0 mmol) and acetone (50 mL) was refluxed for 18 hours. The mixture was cooled, diluted with ether (25 mL) and filtered through Celite. Evaporation of the solvent left the desired benzyl ether 1-G.

Step H To a solution of the olefin from step 1-G (2.0 g, 4.6 mmol) in THF at 0° C. was added 1M borane in THF (14 mL, 14 mmol) and the reaction mixture was stirred for 2 hours. The reaction mixture was treated with trimethylamine-N-oxide (2.15 gm, 19.4 mmol) and the whole was refluxed for 8 hours. The reaction was concentrated in vacuo and the residue was chromatographed on silica gel using 30% EtOAc/Hexane as eluant to yield the desired compound.

Step I To a solution of alcohol 1-H (900 mg, 2.1 mmol) in 20 mL DCM was added triphenylphosphine (1.1 gm, 4.2 mmol) and carbon tetrabromide (1.4 gm, 4.2 mmol). The resulting mixture was stirred for 15 minutes at rt then chromatographed using hexane, then 5% EtOAc/Hexane as eluent to yield the title compound.

Step J Phenol (1.4 g, 15.2 mmol) was added to 95% sodium hydride (365 mg; 14.5 mmol) in DMF (20 mL). After stirring for 30 min, a solution of bromide 1-I (870 mg; 1.69 mmol) in DMF (3 mL) was added and the mixture was stirred at rt for 3 hours. The mixture was poured into excess 1N HCl and extracted with ether. The ether layer was washed with 1N sodium hydroxide twice, dried (MgSO₄), filtered and concentrated in vacuo. The residue was chromatographed on silica gel using 5% ethyl acetate in hexane to yield 630 mg (82%) of 5-benzyloxy-2-(2-phenylethyl)-6-(3-phenoxypropyl)-2,3-dihydrobenzofuran as an oil. H NMR δ 1.86-2.25 (m, 4H), 1.15-2.95 (m, 5H), 3.24 (dd, 1H, J=16 Hz, J=8.5 Hz), 3.96 (t, 3H, J=6.5 Hz), 4.6-4.83 (m, 1H), 4.99 (s, 2H), 6.65 (s, 1H), 6.77 (s, 1H), 6.80-7.0 (m, 2H), 7.1-7.5 (m, 13H).

Step K Boron tribromide solution (1M DCM: 1.9 mL; 1.88 mmol) was added drop-wise to a solution of the benzyl ether from step 1-J in dichloromethane (30 mL) at −78° C. The mixture was stirred for 10 minutes and methanol (0.5 mL) was then added drop-wise. The mixture was brought to rt and saturated sodium bicarbonate solution added. The organic layer was separated, dried (MgSO₄), filtered and concentrated in vacuo. The residue was chromatographed on silica gel using 20% EtOAc/hexanes to yield 5-hydroxy-2-(2-phenylethyl)-6-(3-phenoxypropyl)-2,3-dihydrobenzofuran, m.p. 65-70° C. 1H NMR δ 1.86-2.24 (m, 4H), 2.68-2.95 (m, 5H), 3.2 (dd, 1H, J=15 Hz, J=7.4), 4.40 (t, 2H, J=5.54), 4.67-4.83 (m, 1H), 5.21 (s, 1H), 6.58 (s, 1H), 6.67 (s, 1H), 6.88-7.04 (m, 3H), 7.13-7.39 (m, 7H).

Step L To a solution of the phenol from step 1-K (151 mg, 0.37 mmol) in DMF (12 mL) and cesium carbonate (120 mg, 0.37 mmol) was added tert-butyl bromoacetate (0.05 mL, 0.37 mmol) and the mixture was stirred at rt for 18 hours. The reaction mixture was partitioned between ether and saturated sodium bicarbonate and separated. The organic phase was washed with water 7 times and dried and concentrated. The resulting solid was used directly in the next reaction without further purification.

Step M To a solution of the compound from step 1-L (195 mg, 0.37 mmol) in DCM (2 mL) was added 2 mL of TFA and the mixture was stirred for 1 h at rt. Evaporation of the solvents and reverse phase chromatography afforded the desired compound. 1H NMR δ (CDCl₃, 250 MHz) 1.89-2.23 (m, 4H), 2.71-3.30 (m, 6H), 4.02 (t, 2H, J=7 Hz) 4.58 (s, 2H), 4.76 (m, 1H), 6.64 (s, 1H), 6.66 (s, 1H), 6.91-6.98 (m, 3H), 7.18-7.33 (m, 8H).

### EXAMPLE 2

### {[6-[3-(2chlorophenoxy)propyl)-2-(2-phenylethyl)-2,3-dihydro-1-benzofuran-5-yl]oxy} acetic

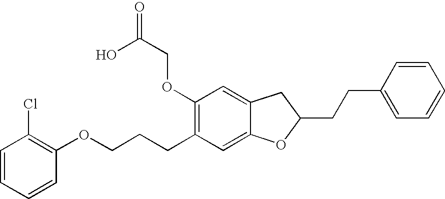

This compound was prepared in a fashion analogous to compound 1 but 2-chlorophenol was substituted for phenol in step 1-J. 1H NMR δ (CDCl₃, 400 MHz) 1.89-2.23 (m, 4H), 2.71-3.30 (m, 6H), 4.02 (t, 2H, J=7 Hz) 4.58 (s, 2H), 4.76 (m, 1H), 6.64 (s, 1H), 6.88-6.98 (m, 3H), 7.2-7.4 (m, 7H).

### EXAMPLE 3

### 2-(2-phenylethyl)-2,3-dihydro-1-benzofuran-5-yl]oxy} acetic acid

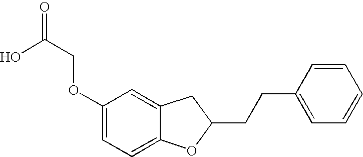

Step A To a solution containing 120 mg (0.5 mmol) of 5-hydroxy-2-(2-phenylethyl)-2,3-dihydrobenzofuran (example 1-D) and 0.088 mL (0.6 mmol) of tert-butyl bromoacetate in 3 mL of THF was added 15 mg (0.6 mmol) of NaH powder. The reaction was stirred for 5 h before it was quenched with water and extracted with ether. Column chromatography (30% EtOAc/Hexanes) prvided the desired ester as an oil. LCMS (M+Na=377.11).

Step B A solution of the ester from step 3-B (71 mg, 0.2 mmol) was dissolved in 2 mL of DCM and treated with 2 mL of TFA. The reaction mixture was stirred for 3 h before it was concentrated and purified by reverse phase chromatography. 1H NMR δ (CDCl3, 400 MHz) 1.85-1.91 (m, 1H), 2.05-2.2 (m, 1H), 2.71-2.91 (m, 3H), 3.25 (dd, 1H), 4.58 (s, 2H), 4.77 (m, 1H), 6.65 (s, 2H), 6.80 (s, 1H), 7.2-7.4 (m, 6H).

### EXAMPLE 4

### 6-{3-[2-chloro-4-(1H-tetrazol-5-yl)phenoxy]propyl}-2-(2-phenylethyl)-2,3-dihydro-1benzofuran-5-oI

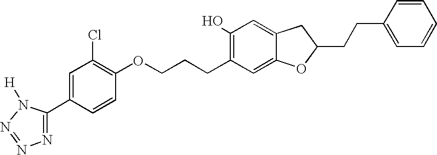

This compound was synthesized according to the method of Lau et al, J. Med. Chem., (1992), 35(7), 1299-318.

Examples 1-4 were found to inhibit BACE cleavage of the full length APP. However, the compounds did not interact with BACE in assays using short peptide substrates.

It was hypothesized that the compounds may interact with the APP substrate rather than the BACE enzyme, or that the compounds may bind non-specifically to the full length APP, rendering the APP protein insoluble and inaccessible to BACE.

In order to determine the mode of inhibition of Examples 1-4, the following assay was developed.

### EXAMPLE 5

A biotinylated APP variant (400 nM) was combined with Examples 1-3 under assay conditions (50 mM sodium acetate, pH 4.5; 1x Protease Inhibitor cocktail (Roche cat # 1836153); 100 μg/mL bovine serum albumin, 0.2% CHAPS). Incubations with vehicle, including DMSO (compound diluent), and incubations lacking the APP variant were carried out as controls for each experiment. Each mixture was incubated at 37° C. for 1 hour.

Thereafter, 70 μL of each incubation mixture was placed in its own well of a 96-well plate. 5 μL of 1 M Tris-Cl, pH 8.0 was added to each well to neutralize any binding reaction.

In Example 5A, a mixture of ALPHA beads was prepared from

(1) 20 μg/mL streptavidin donor beads;

(2) 20 μg/mL Protein A acceptor beads (from AlphaScreen General IgG (Protein A) Detection Kit Perkin Elmer Life Sciences; 10,000 pts (Part Number 6760617M)), containing 150 ng/mL of a rabbit polyclonal antibody directed against the C-terminal region of Aβ; and

(3) 0.1 % bovine serum albumin.

The beads were combined in a phosphate buffered saline.

In Example 5B, a mixture of ALPHA beads was prepared from

(1) 20 μg/mL streptavidin donor beads;

(2) 20 μg/mL Protein A acceptor beads (from AlphaScreen General IgG (Protein A) Detection Kit Perkin Elmer Life Sciences; 10,000 pts (Part Number 6760617M)),20 μg/mL mouse IgG acceptor beads, containing 160 ng/mL of monoclonal antibody LN27, which is directed against the N-terminal region of APP; and

(3) 0.1% bovine serum albumin; were combined in phosphate buffered saline.

In 5A and 5B, 46 μL of the ALPHA bead mixture was combined with 4 μL of each of the incubation mixtures. The beads were incubated overnight, in the dark, at room temperature.

The following day, the plates were read using ALPHAQUEST, to measure the degree of binding of the antibodies.

Compounds that do not interact with APP or APP variant at either of the binding sites of the antibodies of 5A and 5B will produce a measurable signal which is approximately the same as vehicle. Compounds that interact non-specifically with APP or APP variant will cause the APP or APP variant to precipitate from solution. As a result, the protein should not be accessible to any antibody and the measurable signal should be reduced relative to vehicle. Compounds which interact with APP or APP variant near the binding site of one of the antibodies will demonstrate a reduced signal for the antibody proximal to the compound binding site. However, the signal should not be reduced for the antibody at the non-proximal site.

The results of Examples 5A and 5B indicated that the compounds of Examples 1 and 2 interacted with the APP variant within its Aβ domain, proximal to the C-terminal binding site. The compound of Example 3 demonstrated little or no interaction with the APP variant near either antibody binding site.

### EXAMPLE 6

Compounds that bind the APP variant near the C-terminal region of the Aβ domain were also evaluated to determine whether the compounds inhibit gamma-secretase cleavage of APP. Accordingly, Examples 1-4 were tested for their ability to inhibit gamma-secretase cleavage of both a full-length CTFβ substrate, as described in Li, 2000, Proc. Natl Acad Sci. 97:6138-6143. The assays were carried out as follows:

Inhibition of CTFβ Cleavage

Cleavage Reaction:

10 μL 10× protease inhibitor cocktail (Roche, cat # 1836153)
25 μL 4× Buffer (50 mM PIPES pH7.0, 150 mM KCl, 5 mM MgCl₂, 5 mM CaCl₂)
2 μL 10% CHAPSO
5 μL HeLa cell membrane extract characterized for gamma-secretase activity
7 μL 25 μM bacterially expressed CTFβ
51 μL water
1 μL compound in DMSO
   The reactions were incubated at 37° C. for 150 min and quenched with 25 μL 5× RIPA buffer (750mM NaCl, 5% NP-40, 2.5% DOC, 0.5% SDS, 250 mM Tris H8.0).
   Detection:

Gamma-secretase cleavage of the CTFβ substrate was assessed by incubating 50 μL of the reaction with 25 μL of 4 μg/mL biotinylated antibody 6E-10 which is specific for the N-terminal region of Aβ, and 25 μL of 1.5 μg/mL of a ruthinylated antibody G2-10, which is specific to the C-terminal region of Aβ40. The C-terminal region is present only after gamma secretase cleavage of Aβ. The antibodies were allowed to bind overnight at room temperature. The following day, 25 μL of 80 μg/mL streptavidin-coated Dynabeads (M-280) were added to each reaction, allowed to bind for 30 minutes, and quenched with 275 μL IGEN assay buffer. Quantification of the gamma-secretase cleavage products was carried out using an Origen Analyzer.

Results:

Examples 1-4 were assayed for inhibition of gamma-secretase cleavage of CTFβ.

| | CTFβ CLEAVAGE |
|---|---|
| | AT |
| EXAMPLE | POSITION 40 (μM) |
| 1 | 29 |
| 2 | 8 |
| 3 | 49 |
| 4 | ND |

As shown above, each of Examples 1-3 inhibit cleavage of CTFβ by gamma secretase.

### EXAMPLE 7

Compounds interacting with secretase or antibody binding to the Aβ domain of APP (or an APP variant) nay also be expected to interact with Aβ or related amyloidogenic peptides. An Aβ variant peptide similar to Aβ 1-40, but with Glu and Val replacing the N-terminal two amino acids (hereafter referred to as “EV-amyloid”), as described in WO 02/094985 and US 2003/0200555 A1, was used to assess interactions between benzofurans and amyloid peptide. As with full length APP variant in Example 5, masking of antibody binding sites was used to assay the potential interactions.

The assay was carried out as follows:

1. A master mix of EV-amyloid peptide in binding buffer was made as follows: 10650 μL 0.1% bovine serum albumin in phosphate buffered saline, 1200 μL 10× Protease Inhibitor cocktail (Roche, cat # 1836153), 30 μL EV amyloid peptide (4 μM in DMSO).
2. A negative control mix of binding buffer was prepared by combining 0.1% bovine serum albumin in phosphate buffered saline, 10× Protease inhibitor cocktail, and DMSO in the same ratio.
3. 99 μL of the master mix and the negative control mix were distributed to individual wells in a 96-well plate.
4. 1 μL of Examples 1-4 in DMSO or DMSO alone (vehicle) was added to each well.
5. The binding reaction was then incubated at 37° C. for 1 hour.
6. 20 μL of each reaction was transferred to a second assay plate. Interaction between EV amyloid and antibody was assessed by quantification of the EV amyloid peptide by antibody sandwich assay using ALPHA detection.
7. For EV-amyloid detection, the following two solutions were prepared:
   Solution 1: 24 μL streptavidin donor beads (5 mg/mL, from AlphaScreen General IgG (Protein A) Detection Kit Perkin Elmer Life Sciences; 10,000 pts (Part Number 6760617M)), 2.3 μL of a biotinylated antibody G2-10 (which is specific to the C-terminal region of AD), 1173.7 μL 50 mM Hepes pH 7.5.
   Solution 2: 24 μL Protein A acceptor beads (5 mg/mL from AlphaScreen General IgG (Protein A) Detection Kit Perkin Elmer Life Sciences); 10,000 pts (Part Number 6760617M)), 10.4 μL of an antibody which specifically interacts with NH₂-EVEFR at the amino terminus of the EV amyloid, 1165.6 μL 50 mM Hepes pH 7.5.

The solutions were incubated at room temperature for 1 hour prior to addition to the EV-amyloid binding reaction.
8. To each well in the 96-well plate containing EV amyloid binding reactions, 10 μL of solution 1, 10 μL of solution 2, and 10 μL of 0.1% BSA in PBS was added.
9. The plate was incubated overnight, in the dark, at room temperature, and antibody binding to EV-amyloid was detected via ALPHA Quest.

Suppression of antibody binding to EV-amyloid was observed with an antibody binding to Aβ. Examples 1 and 2, both of which inhibit both BACE processing of APP and gamma-secretase processing of CTFβ, and both of which block the antibody binding to APP, inhibit approximately 40% of antibody binding to EV amyloid. Example 3, which neither blocked BACE processing of APP nor blocked the antibody interaction with APP, had no effect on antibody interactions with EV-amyloid.

### EXAMPLE 8

The BACE2 enzyme cleaves APP at several different positions within the Aβ domain. Differential inhibition of BACE2 cleavage at the different cleavage sites within the Aβ domain of APP would help demonstrate that inhibition is due to compound interaction with the substrate rather than the cleaving enzyme.

Inhibition of BACE2 Cleavage of APP

The effect of Examples 1-4 on cleavage of APP at the beta-secretase cleavage site was assayed as follows:

Cleavage Reaction:

25 μL 0.2 M Sodium Acetate, pH 4.5
10 μL 10× protease inhibitor cocktail (Roche, cat # 1836153)
10 μL Bovine Serum Albumin (1 mg/mL solution)
4 μL 5% CHAPS
1 μL EDTA (150 mM, pH 4.5)
2 μL DF
4 μL MBP(Maltose binding protein)—biotininylated APP fusion protein containing the NFEV BACE cleavage site (bacterially expressed, 10 μM solution)
1 μL DMSO or compound dissolved in DMSO
2 μL BACE2 (CHO cell expressed protein, 250 nM)
41 μL distilled water
   The reactions were incubated at 37° C. for 30 minutes, and were then quenched with 4 μL 1 M TrisHCl, pH 8.0.
   Detection of MBP Biotin APP(NF) Cleavage Product using ALPHA
   ALPHA Assay Mix was Prepared as Follows:
20 μL Streptavidin Donor Beads (from AlphaScreen General IgG (Protein A) Detection Kit Perkin Elmer Life Sciences); 10,000 pts (Part Number 6760617M)
20 μL Protein A Acceptor beads (from AlphaScreen General IgG (Protein A) Detection Kit Perkin Elmer Life Sciences); 10,000 pts (Part Number 6760617M)
3.7 μL Affinity purified anti-NF antibody (203 μg/mL) (described in WO 02/094985)
4.5 mL 0.1% BSA in PBS
4 μL of quenched reaction mixture was combined with 46 μL of the assay mix. The reactions were incubated at room temperature overnight and read using the ALPHAQuest plate reader.

Results:

| EXAMPLE | IC50 (μM) |
|---|---|
| 1 | 9 |
| 2 | 4 |
| 3 | ˜100 |
| 4 | 20 |

Example 3 was inactive against BACE1 and weakly active against BACE2. Examples 1, 2 and 4 were capable of inhibiting BACE2 cleavage of APP at the beta-secretase position with similar effectiveness to their inhibition of BACE1 cleavage at the same site.

### EXAMPLE 9

The effect of inhibition of BACE 2 cleavage of EV amyloid was assayed using a protocol described in Shi et al, 2003, J. Biol. Chem., 278:21286-21294. The results of assaying Examples 1-4 at 100 μM are shown below:

| | INHIBITION OF BACE2 | STIMULATION OF | |
|---|---|---|---|
| | CLEAVAGE OF EV 1-40 | BACE2 CLEAVAGE OF | |
| | AT | EV 1-40 AT | |
| EXAMPLE | 34 site | 19 site | 20 site |
| 1 | +++ | NS | NS |
| 2 | ++++ | NS | NS |
| 3 | ++ | NS | NS |
| 4 | ++++ | + | +++ |

− No effect.
NS—no signal

All four compounds tested disrupted BACE 2 cleavage at position 34. Effects on cleavage at position 19 and 20 could not be determined with Examples 1, 2 and 3, due to a very weak signal. However, Example 2 substantially enhanced cleavage at positions 19 and 20, demonstrating position-dependent effects on BACE2 cleavage of APP.

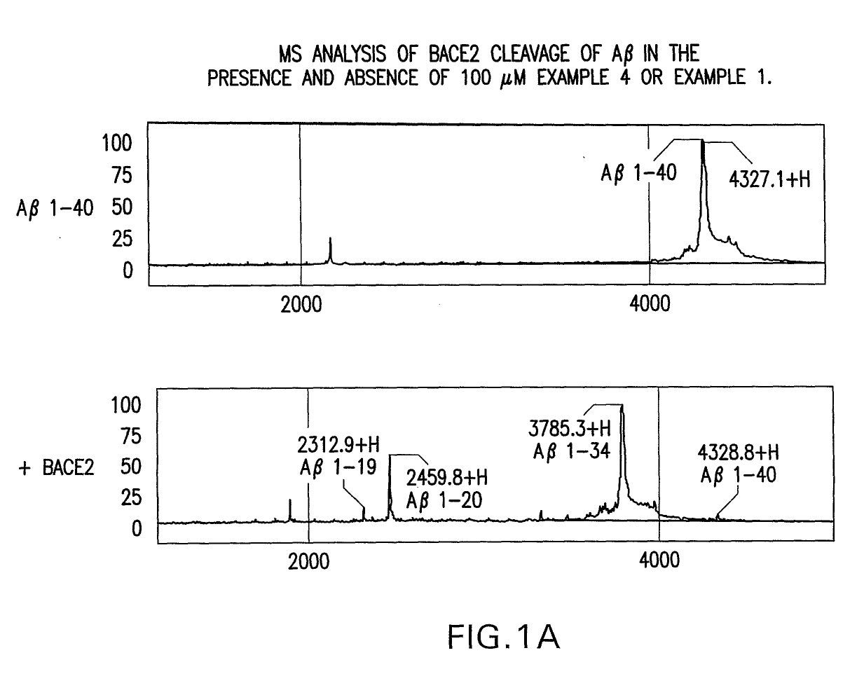

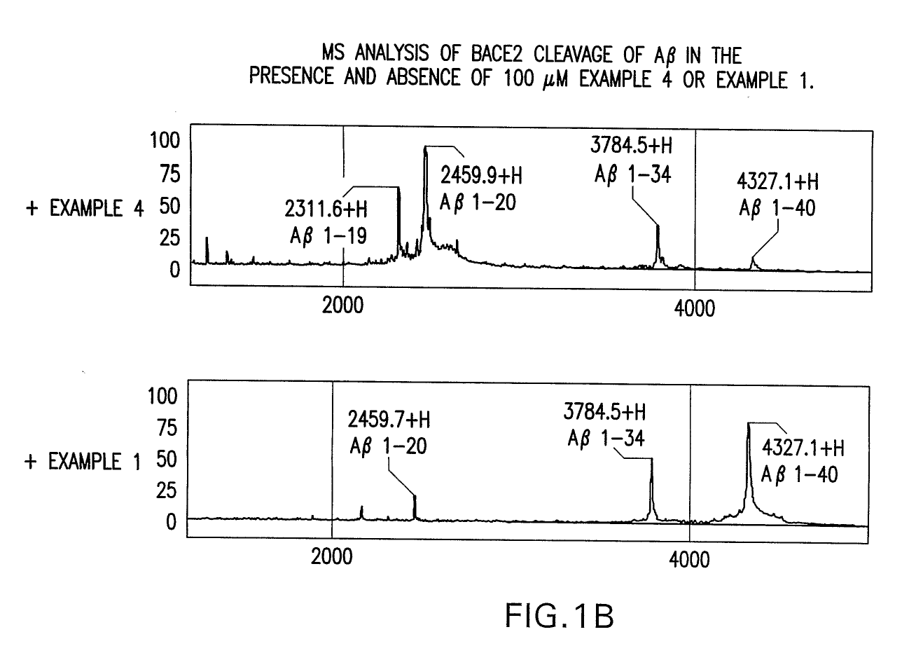

FIG. 1 shows the mass spectrometric analysis of BACE2 cleavage of Aβ in the presence and absence of 100 μM of the compounds of Example 4 and Example 1. Example 4 enhanced cleavage of Aβ at positions 19 and 20, but blocked cleavage at position 34, while Example 4 suppressed cleavage at all Aβ sites. This data demonstrates that the compounds of the invention may act as modulators of secretase cleavage.

### EXAMPLE 10

A bacterially expressed APP variant containing an enhanced BACE cleavage sequence was incubated with Example 2 and BACE 1 protein for 1 h at 37° C. to conduct a Western blot analysis. The reaction was run on a gel and blotted. The BACE-specific cleavage product was detected using an antibody specifically recognizing the N-terminal product arising after BACE cleavage.

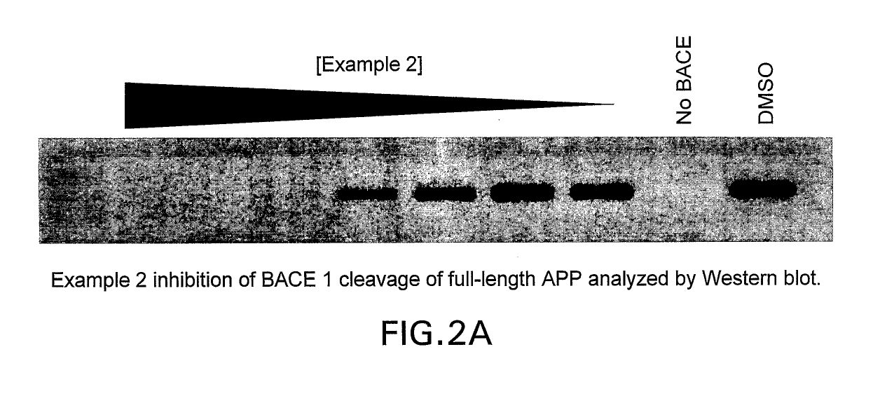

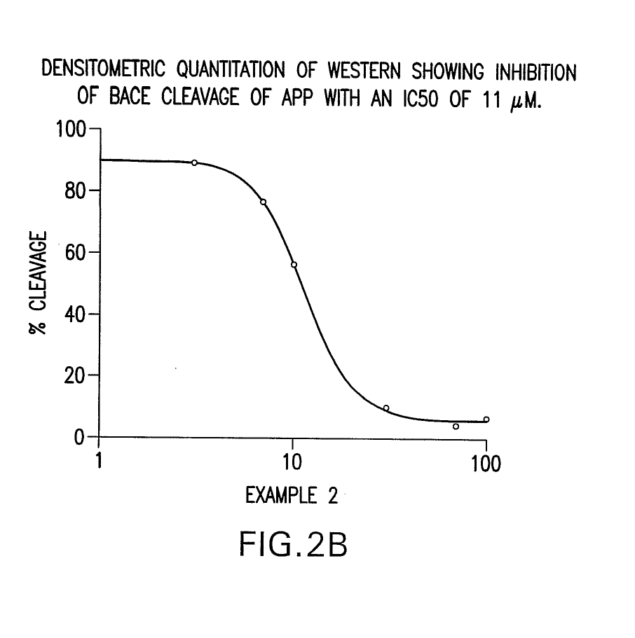

FIG. 2(A) depicts the Western blot. FIG. 2(B) is a densitometric quantitation of the Western blot, showing inhibition of BACE cleavage of APP with an IC50 of 11 μM.

### EXAMPLE 11

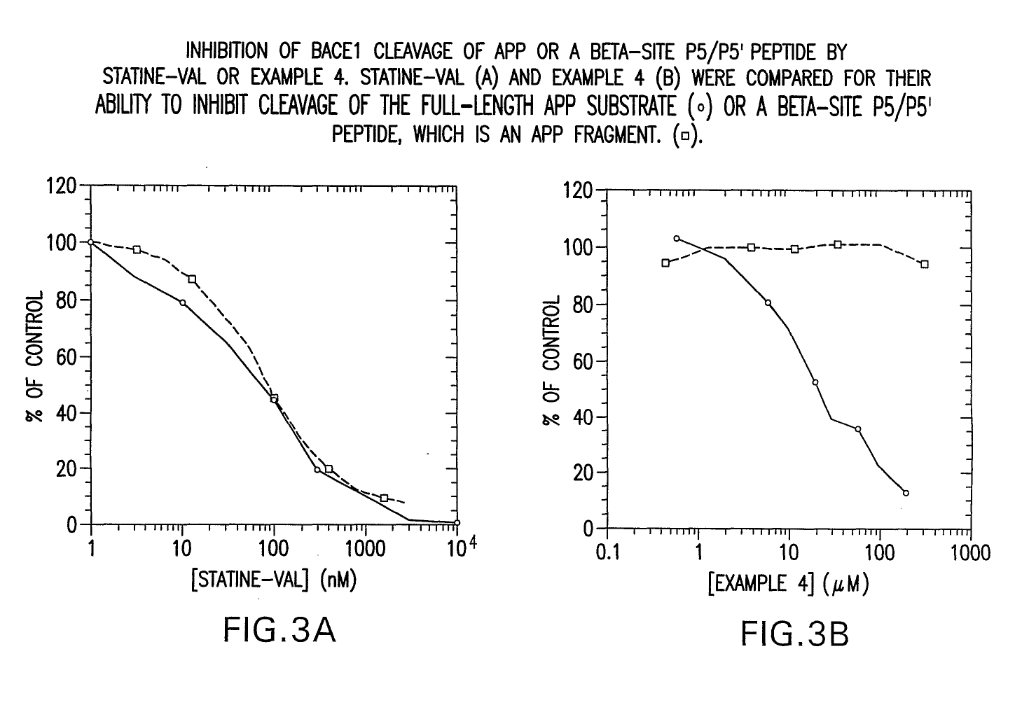

FIG. 3 demonstrates the inhibition of BACE1 cleavage of full-length APP and a beta-site P5/P5′ peptide, an APP fragment, by Statine-Val and Example 4. FIG. 3(A) depicts inhibition by Statine-Val, which is a known inhibitor of BACE cleavage of both APP and APP fragments, including the beta-site P5/P5′ peptide fragment. FIG. 2(B) depicts inhibition by Example 4. The graphs depict inhibition of cleavage of a full-length APP substrate (∘) or the beta-site P5/P5 fragment. (□). Enzymatic activity is shown relative to DMSO-treated BACE cleavage. Statine-Val demonstrations inhibition of cleavage of both APP and the peptide-based substrates, while Example 4 inhibits cleavage of APP only.

### EXAMPLE 12

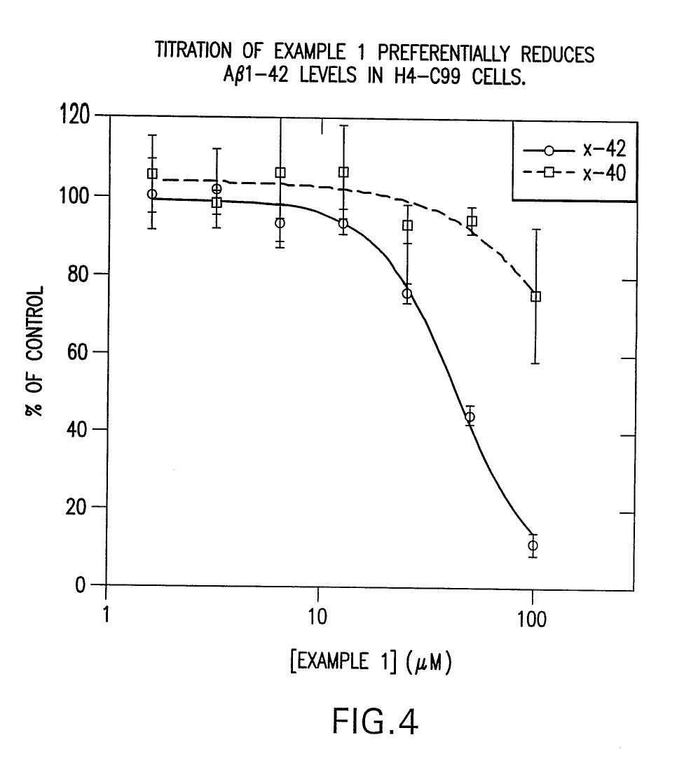

The compound of Example 1 was titrated into H4 neuroglioma cells expressing CTF99. The cells were treated with the compound of Example 1 for 20 h and conditioned media was harvested. Levels of secreted amyloid x-42 and x-40 were assayed via ECL (Li et al, 2000, Proc. Nat'l Acad. Sci. 97:6138-6143, and the production of Aβ42 and Aβ40 were measured and graphed, as shown in FIG. 4. The results demonstrate that the compounds of the invention modulate the production of Aβ peptides to decrease the production of Aβ42 relative to the production of Aβ40.

### EXAMPLE 13

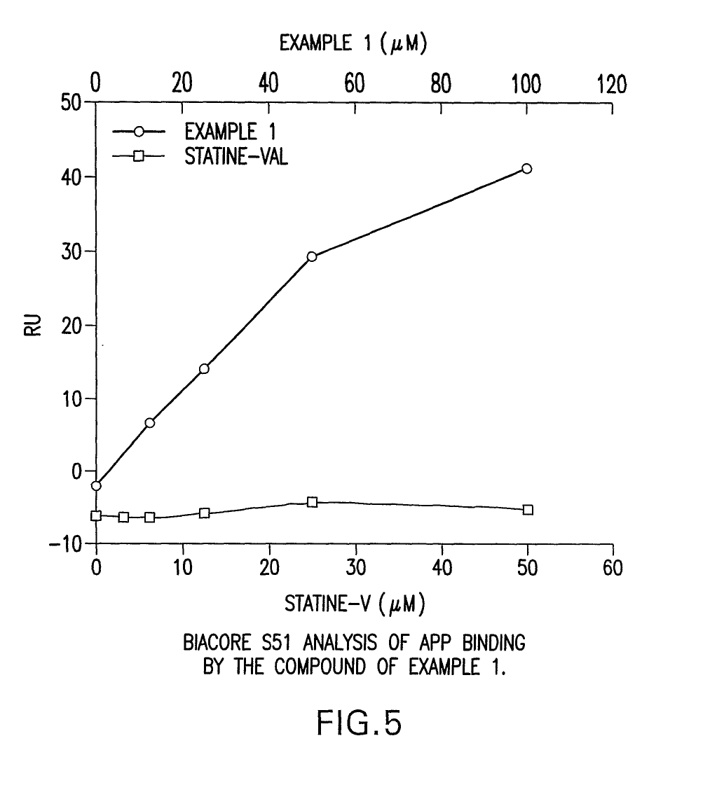

The compounds of formula (I) are believed to inhibit APP cleavage function by binding APP. The binding of the compound of Example 1 to APP was assayed by BiacoreS51. Biotinylated APP and biotinylated sAPPβ were immobilized to a SA-coated sensor chip. A titration of Example 1 was injected and the binding response for each concentration of Example 1 was obtained for both proteins. FIG. 5 is a graph of response units for Compound 2 (◯) or the BACE 1 inhibitor Statine-Val ( [symbol] ) binding to APP.

The data indicate that the compound of Example 1 binds APP but does not bind sAPPβ, and that the BACE inhibitor, Statine-Val, binds neither APP nor sAPPβ. The ability of the compound of Example 1 to bind APP combined with its inhibition of APP processing by several different enzymes indicates that the inhibition of APP processing may be caused by an interaction between the compound and APP rather than between the compound and enzyme.

The following abbreviations are used throughout the text:
Me: methyl
Et: ethyl
Bu: butyl
tBu: tert-butyl
Ar: aryl
Bn: benzyl
THf: tetrahydrofuran
TFA: trifluoroacetic acid
TPP: triphenyl phosphate
DMF: N,N′-dimethyl formamide
DCM: dichloromethane
BSA: bovine serum albumin
CHAPSO: 3-[(cholamidopropyl)dimethylammonio]-2-hydroxy-1-propanesulfonate
CHAPS: 3-[(cholamidopropyl)dimethylammonio]-1-propanesulfonate
DMSO: dimethylsufoxide
RIPA: radioimmunoprecipitation assay
PIPES: piperazine-N,N′-bis(ethanesulfonic acid)
PBS: phosphate buffered saline
EDTA: ethylene diamine tetraacetic acid

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, reaction conditions other than the particular conditions as set forth herein above may be applicable as a consequence of variations in the reagents or methodology to prepare the compounds from the processes of the invention indicated above. Likewise, the specific reactivity of starting materials may vary according to and depending upon the particular substituents present or the conditions of manufacture, and such expected variations or differences in the results are contemplated in accordance with the objects and practices of the present invention. It is intended, therefore, that the invention be defined by the scope of the claims which follow and that such claims be interpreted as broadly as is reasonable.

It is further to be understood that all values are approximate, and are provided for description. Patents, patent applications, publications, product descriptions, and protocols are cited throughout this application, the disclosures of which are incorporated herein by reference in their entireties for all purposes.

1. A process for determining the interaction of a substance to amyloid precursor protein (APP) or amyloid β peptide (Aβ), said method comprising
(a) forming a first test solution comprising (i) APP or APP variant having an sAPPβ region and an Aβ region, (ii) a substance, and (iii) a first antibody, wherein the first antibody is specific to the sAPPβ region of the APP or APP variant, under conditions favoring formation of an sAPPβ-antibody complex, to give a first test signaling reading of the sAPPβ-antibody complex;
(b) forming a second test solution comprising (i) the APP or APP variant having an sAPPβ region and an Aβ region, (ii) the substance, and (iii) a second antibody, wherein the second antibody is specific to the Aβ region of the APP or APP variant, under conditions favoring formation of an Aβ-antibody complex, to give a second test signaling reading of the Aβ-antibody complex;
(c) forming a control solution comprising (i) the APP or APP variant having an sAPPβ region and an Aβ region, and (ii) the first and second antibodies, under conditions favoring formation of an sAPPβ-antibody complex and an Aβ-antibody complex, to give a first control signaling reading of an sAPPβ-antibody complex and a second control signaling reading of an Aβ-antibody complex;
(d) comparing the first test signaling reading and the first control signaling reading, and comparing the second test signaling reading and the second control signaling reading,
wherein:
(i) if the first test signaling reading is equal to the first control signaling reading and the second test signaling reading is equal to the second control signaling reading, then the substance does not interact with APP,
(ii) if the first test signaling reading is lower than the first control signaling reading and the second test signaling reading is equal to the second control signaling reading, then the substance binds specifically to the sAPPβ region of the APP,
(iii) if the first test signaling reading is equal to the first control signaling reading and the second test signaling reading is lower than the second control signaling reading, then the substance binds specifically to the Aβ region of the APP.

2. The process of claim 1, wherein each of the test solutions and the control solution further comprises a diluent.

3. The process of claim 1, wherein the test solutions and the control solution further comprises a pH control agent.

4. The process of claim 1, wherein the substance has a molecular weight below about 2 kD.

5. The process of claim 1 wherein the APP is detectably labeled with a ligand.

6. The process of claim 1 wherein the test solutions and the control solution comprise an acceptor bead.

7. The process of claim 1 wherein the second antibody is specific to amino acids 1-17 or 28-40 of Aβ.

8. The process of claim 1 wherein the second antibody is specific to the C-terminus of the Aβ region.

9. The process of claim 1 wherein the second antibody is specific to the N-terminus of the Aβ region.

10. The process of claim 1 wherein the APP or APP variant is an APP variant.

11. A process for determining the interaction of a substance to amyloid β peptide (Aβ), said method comprising
(a) forming a first test solution comprising (i) APP or APP variant having an sAPPβ region and an Aβ region, said Aβ region having a first sub-region and a second sub-region, and (ii) the substance, and (iii) a first antibody, wherein the first antibody is specific to the first sub-region of the Aβ region of the APP or APP variant, under conditions favoring formation of an Aβ-antibody complex, to give a first test signaling reading of the Aβ-antibody complex;
(b) forming a second test solution comprising (i) the APP or APP variant having an sAPPβ region and an Aβ region having the first sub-region and the second sub-region, (ii) the substance, and (iii) a second antibody, wherein the second antibody is specific to the second sub-region of the Aβ region of the APP or APP variant, under conditions favoring formation of an Aβ-antibody complex, to give a second test signaling reading of the Aβ-antibody complex;
(c) forming a control solution comprising (i) the APP or APP variant having an sAPPβ region and an Aβ region having the first sub-region and the second sub-region, and (ii) the first and second antibodies, under conditions favoring formation of an Aβ-antibody complex, to give a first control signaling reading of an Aβ-antibody complex and a second control signaling reading of an Aβ-antibody complex;
(d) comparing the first test signaling reading and the first control signaling reading, and comparing the second test signaling reading and the second control signaling reading,
wherein:
(i) if the first test signaling reading is equal to the first control signaling reading and the second test signaling reading is equal to the second control signaling reading, then the substance does not interact with Aβ,
(ii) if the first test signaling reading is lower than the first control signaling reading and the second test signaling reading is equal to the second control signaling reading, then the substance binds specifically to the first sub-region of Aβ,
(iii) if the first test signaling reading is equal to the first control signaling reading and the second test signaling reading is lower than the second control signaling reading, then the substance binds specifically to the second sub-region of Aβ.

12. The process of claim 11, wherein the first sub-region of Aβ is the C-terminus, and the second sub-region of Aβ is the N-terminus.

13. The process of claim 11, wherein each of the test solutions and the control solution further comprises a diluent.

14. The process of claim 11, wherein each of the test solutions and the control solution further comprises a pH control agent.

15. The process of claim 11, wherein the APP or APP variant is an APP variant.

16. The process of claim 11, wherein the substance has a molecular weight below about 2 kD.

17. The process of claim 11 wherein the APP or APP variant is detectably labeled with a ligand.

18. The process of claim 11 wherein the test solutions and the control solution comprise an acceptor bead.

19. A substance which interacts with APP or Aβ, the structure of which is identified by the assay of claim 1.

20. A substance which interacts with APP or Aβ, the structure of which is identified by the assay of claim 11.

21. A compound of formula (I) 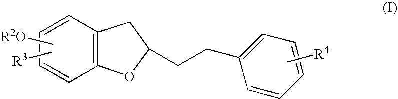
wherein
R⁴ is optionally present and is selected from the group consisting of
(a) halogen,
(b) C₁₋₆ alkyl, and
(c) C₁₋₆ alkoxy;
R² is selected from the group consisting of
(a) hydrogen,
(b) (CH₂)ₘ—C(═O)—OR⁵ wherein R⁵ is selected from the group consisting of hydrogen and C₁₋₆ alkyl;
m is 1, 2 or 3;
R³ is selected from the group consisting of
(a) hydrogen, and
(b) 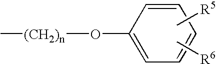
wherein R⁵ and R⁶ are selected from the group consisting of
(i) hydrogen,
(ii) halogen,
(iii) C₁₋₆ alkyl,
(iv) C₁₋₆ alkoxy, and
(v) tetrazolyl,
n is 3, 4 or 5,
or a pharmaceutically acceptable salt thereof.

22. A compound of claim 21 wherein R³ is

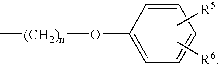

23. A method of treating Alzheimer's Disease in a mammal in need thereof, said method comprising administering to the mammal a therapeutically effective amount of a compound of claim 21, or a pharmaceutically acceptable salt thereof.
